(19) United States
(12) Patent Application Publication
(10) Pub. No.: US 2023/0181122 A1
(43) Pub. Date: Jun. 15, 2023

(54) STRATIFICATION ENGINE FOR PHARMACOGENOMIC TESTING

(71) Applicant: OneOme LLC, Minneapolis, MN (US)

(72) Inventor: Tyler Koep, Minneapolis, MN (US)

(21) Appl. No.: 18/064,380

(22) Filed: Dec. 12, 2022

Related U.S. Application Data
(60) Provisional application No. 63/288,143, filed on Dec. 10, 2021.

(51) Int. Cl. A61B 5/00; G16H 20/10; G16H 50/30

(52) U.S. Cl. CPC A61B 5/7275; G16H 20/10; G16H 50/30

(57) ABSTRACT

A pharmacogenomic stratification engine can be used to identify patients within a given population or plan with the greatest probability for pharmacogenomic (PGx) testing to improve health outcomes, and/or avoid non-optimized therapies. A patient, sometimes referred to as a ‘member,’ that is taking or has taken pharmaceutical and/or therapeutic compound(s) can be assigned a unique PGx risk score from the stratification engine and thereafter grouped into very high, high, medium, low, and very low risk categories correlating to expected benefit of PGx testing. The stratification engine can be capable of operating on disparate information from a variety of data sources (e.g. from various pharmacy benefits managers, etc.) and in one form is created at least in part through use of advanced machine learning.

### RELATED APPLICATIONS

The present application claims priority to and incorporates by reference hereto U.S. Provisional Patent Application No. 62/288,143 filed on Dec. 10, 2021 of the same title.

### BACKGROUND OF THE INVENTION

### Technical Field

The present disclosure generally relates to pharmacogenomic testing, and more particularly, but not exclusively, to recommendations for pharmacogenomic testing through use of machine learning.

### Background

Providing pharmacogenomic testing (PGx) for populations receiving medication remains an area of interest. Some existing systems have various shortcomings relative to certain applications.

Non-optimized therapy, or non-optimized medication therapy, is a broad term that encompasses a number of concepts that together indicate that a patient is undergoing a course of treatment for a medical condition that is not optimal and can lead to negative consequences. Non-optimized therapy is often associated with treatment by medication, where non-optimization can result from non-adherence to prescribed/recommended treatment protocols, inappropriate prescriptions (including incorrect dosage), medications that cause secondary health issues, treatment failure (TF), new medical problems (NMPs), or other issues.

In a recent study, non-optimized medication therapy was estimated to cost the United States $528.4 billion, approximately 16% of total healthcare expenditures in 2016, due to increased morbidity and mortality.

Non-optimized therapy can lead to the following negative outcomes, driving the bulk of the $528.4 billion estimate:
Physician visit
Additional treatment
Emergency department visit
Hospital admission
Long term care admission
Death

Accordingly, a need exists to identify patients that are on a non-optimized track as soon as possible before the negative effects described above take effect or the effects can be minimized, and direct the patient to a therapy that is more likely to put the patient on a beneficial therapeutic track. Additionally, even if a patient appears to be a stable track a method of assessing patients likely to benefit from PGx testing is needed. The foregoing includes identification of candidates most likely to benefit from PGx testing, which can more accurately identify in those patients the most beneficial therapies. There remains a need for further contributions in this area of technology.

### SUMMARY OF THE INVENTION

One embodiment of the present disclosure is a unique stratification engine useful to recommend potential pharmacogenomics testing. Other embodiments include apparatuses, systems, devices, hardware, methods, and combinations for stratifying member populations for purposes of pharmacogenomic testing. Further embodiments, forms, features, aspects, benefits, and advantages of the present application shall become apparent to those of ordinary skill in the art from the description and figures provided herewith.

### BRIEF DESCRIPTION OF THE FIGURES

FIG. 1 is a block diagram depiction of an embodiment of a stratification engine pipeline.

FIG. 2 depicts an embodiment of a computer structured to build/execute/develop a stratification engine model.

FIG. 3 is a block diagram depiction an embodiment of a stratification engine.

FIG. 4 depicts results from execution of a stratification engine.

### DETAILED DESCRIPTION OF THE INVENTION

For the purposes of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

The present stratification engine aims to identify members/patients, within a given population or plan, with the greatest probability for pharmacogenomic (PGx) testing to improve non-optimized therapy, or to otherwise provide beneficial results.

As described further below, each member is given a unique PGx risk score and grouped into very high, high, medium, low, and very low risk categories correlating to expected benefit of PGx testing. As used herein, a “member” refers to an individual person that is a patient/recipient/consumer of, or candidate for, a pharmaceutical and/or therapeutic compound. The member can, but need not, be a current participant in a health insurance and/or pharmacy insurance plan. For illustrative purposes, the examples below reference a member of a health insurance plan that receives a medication prescription and from which further information can be assessed by comparing the member to a grouping of other similarly situated individuals. As will be appreciated by those of skill in the art, no limitation is hereby intended that such a member must be a current member of the plan, or that the medication prescription is for a controlled drug that can only be prescribed by a licensed pharmacist, or even that the cohort to which the member is compared to is from the same insurance plan, etc. The stratification engine described herein is capable of operating on disparate information from a variety of data sources (e.g. from various pharmacy benefits managers, etc) and can include prescribed or over-the-counter medications, or controlled and non-controlled substances.

The unique PGx risk score generated by the present invention seeks to identify and categorize those members with the greatest expected beneficial impact from PGx testing, wherein the higher the risk scores the greater the likelihood the member is on a potentially harmful non-optimized therapy track. Thus, the PGx test is most likely to be of benefit to the “very high” risk cohort, whereby the test is most likely to identify a more optimized therapy track for this cohort, or otherwise identify candidates likely to benefit from testing regardless of they are on a non-optimized path or not. The present invention is likely to be of benefit to various partners, including pharmacy benefits managers, health insurance providers, hospital systems, etc. Descriptive outcomes provided by the stratification engine described herein can include such items as number of medications per person, number of PGx actionable medications per person, and demographic findings will be presented to the partner. Demographic factors can include age, sex, race, physical characteristics, or business related characteristics such as the type of insurance the member has (Medicare, Medicaid, or private insurance), as well as other factors of relevance. Additional data entries provided in advance, described below, may be used to demonstrate preliminary correlations between PGx actionability scores and members.

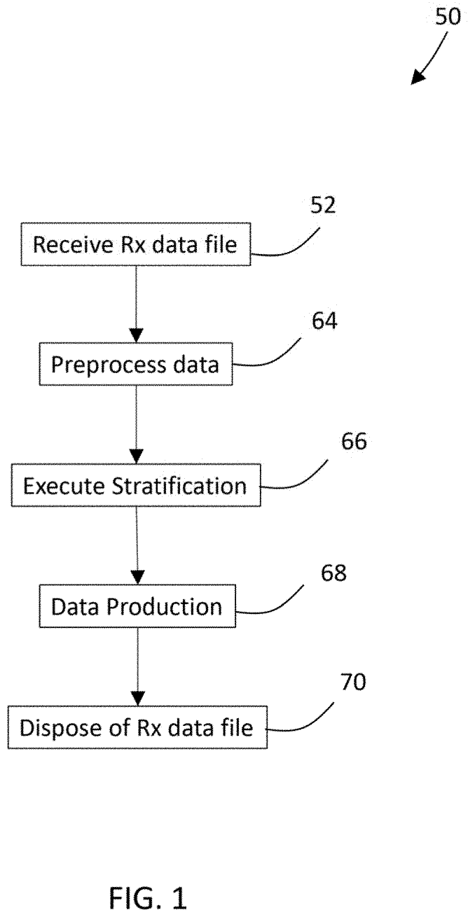

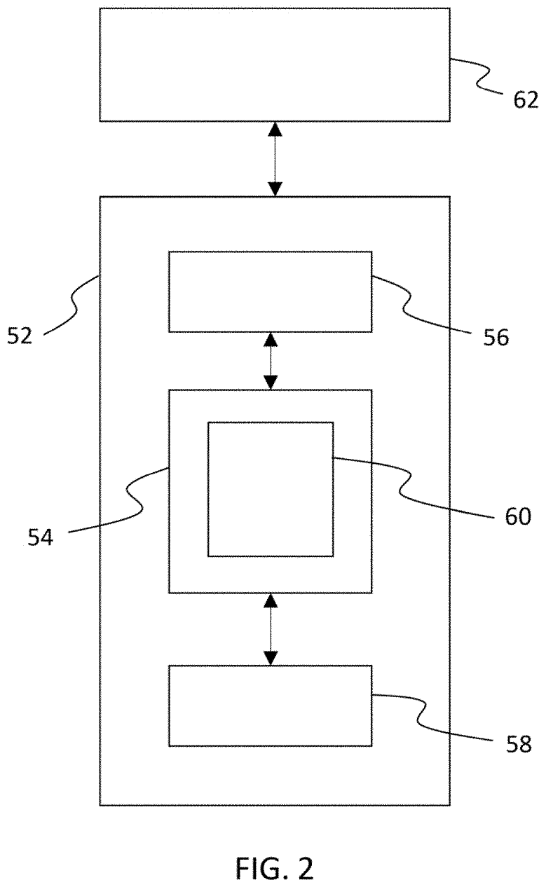

With reference to FIGS. 1 and 2, the stratification tool 50 is described. The stratification tool 50 includes a pipeline through which data can be acquired and processed to produce a prediction for which action can be taken with respect to a member. The stratification tool 50 can be executed in one or more computers 52 capable of receiving and processing relevant data. With specific reference to FIG. 2, a computer 51 can include a processing device 54, an input/output device 56, memory 58, and operating logic 60. Furthermore, computer 51 can be configured to communicate with one or more external devices 62.

The input/output device 56 may be any type of device that allows the computer 51 to communicate with the external device 62. For example, the input/output device may be a network adapter, network card, or a port (e.g., a USB port, serial port, parallel port, VGA, DVI, HDMI, FireWire, CAT 5, or any other type of port). The input/output device 56 may be comprised of hardware, software, and/or firmware. It is contemplated that the input/output device 56 includes more than one of these adapters, cards, or ports.

The external device 62 may be any type of device that allows data to be inputted or outputted from the computer 51. To set forth just a few non-limiting examples, the external device 62 may be another server, a printer, a display, an alarm, an illuminated indicator, a keyboard, a mouse, mouse button, or a touch screen display. In some forms, there may be more than one external device in communication with the computer 51. Furthermore, it is contemplated that the external device 62 may be integrated into the computer 51. In such forms, the computer 51 can include different configurations of computers 51 used within it, including one or more computers 51 that communicate with one or more external devices 62, while one or more other computers 51 are integrated with the external device 62.

Processing device 54 can be of a programmable type, a dedicated, hardwired state machine, or a combination of these; and can further include multiple processors, Arithmetic-Logic Units (ALUs), Central Processing Units (CPUs), or the like. For forms of processing device 54 with multiple processing units, distributed, pipelined, and/or parallel processing can be utilized as appropriate. Processing device 54 may be dedicated to performance of just the operations described herein or may be utilized in one or more additional applications. In the depicted form, processing device 54 is of a programmable variety that executes algorithms and processes data in accordance with operating logic 60 as defined by programming instructions (such as software or firmware) stored in memory 58. Alternatively or additionally, operating logic 60 for processing device 54 is at least partially defined by hardwired logic or other hardware. Processing device 54 can be comprised of one or more components of any type suitable to process the signals received from input/output device 56 or elsewhere, and provide desired output signals. Such components may include digital circuitry, analog circuitry, or a combination of both.

Memory 58 may be of one or more types, such as a solid-state variety, electromagnetic variety, optical variety, or a combination of these forms. Furthermore, memory 58 can be volatile, nonvolatile, or a mixture of these types, and some or all of memory 58 can be of a portable variety, such as a disk, tape, memory stick, cartridge, or the like. In addition, memory 58 can store data that is manipulated by the operating logic 60 of processing device 54, such as data representative of signals received from and/or sent to input/output device 56 in addition to or in lieu of storing programming instructions defining operating logic 60, just to name one example.

In one embodiment the computer 51 receives an Rx data file from one or more sources such as a pharmacy benefits manager, health insurance provider, etc., upon which the stratification tool 50 operates. This process can be carried out by an operator, which may include one or more persons and/or automated routines capable of undertaking the step at 52 and/or any subsequent step. For conciseness, reference will be made to “operator” below with respect to a variety of steps, but it will be appreciated that one or more steps can be executed by different people and/or automated routines, etc. Such receipt of data by the computer 51 can take a variety of forms including physical media delivery (CD-ROM, memory stick, hardcopy, etc) or electronic delivery (e.g. SFTP, cloud based deposit and delivery, etc), etc. To integrate data for use by the stratification tool 50 the operator may need to preprocess the data at step 52, which can include cleaning the data file by removing extraneous entries, correcting misspellings, deleting erroneous data, formatting the data, performing integrations with other data, etc. In some forms, which include delivery of hardcopy, scanning may be used to produce an equivalent electronic record, with any associated further processing to correct errors, etc. The Preprocessing data step 64 can be referred to as data munging or data wrangling and is a sometimes necessary step if the received Rx data file at provided to the computer 51 is formatted and/or populated with data inconsistent with subsequent pipeline activities.

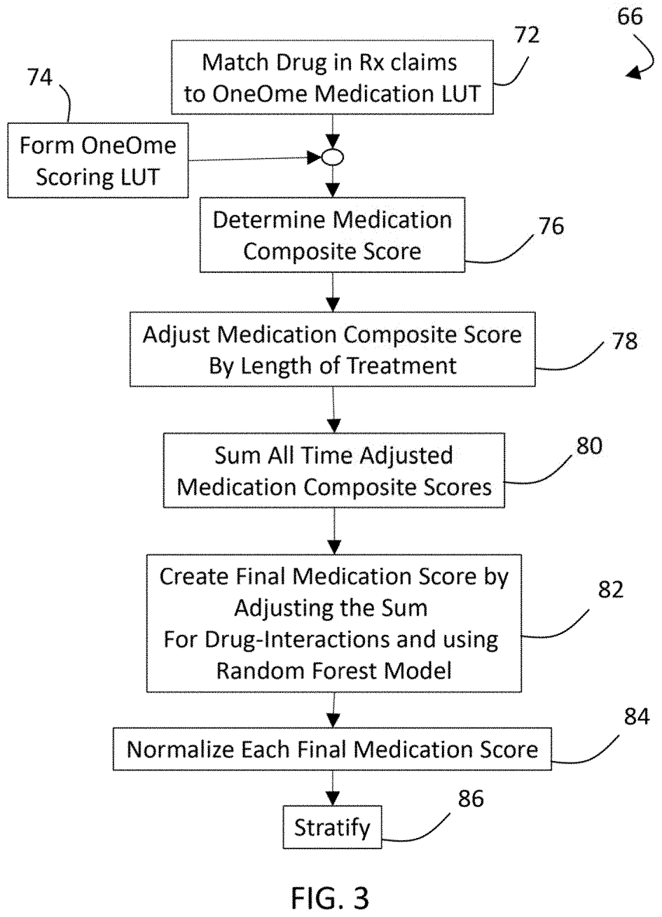

Once properly formatted the operator can execute the stratification engine at step 66, which is described further below with respect to FIG. 3. The stratification engine is used to interpret the data set results received at step 52 so that a recommendation can be made for one or more members taking a particular medication, or combination of medications, as indicated by the date file processed in step 52, as to the likelihood of the member benefiting from PGx testing. Based thereon, the stratification engine determines a unique PGx risk score for each member, and/or creates groups of members, wherein the PGx risk score can be segmented into very high, high, medium, low, and very low risk categories correlating to expected benefit of PGx testing for the member. As will be appreciated, such categories of “very high,” “high,” “medium,” “low,” and “very low” are just one example of a stratification technique. In other embodiments, fewer strata can be used and/or other labels provided to each strata.

Once the stratification is determined and the strata within which the individual member resides, data can be produced at step 68 which can take a variety of forms, including for example a report/chart annotation, etc useful for the individual member's awareness and/or for an associated medical professional to use to consult with the member for taking a particular action with regard to the members medical treatment. The data production at step 68 can also include an alarm/notification/text/etc useful to bring attention to the availability of the produced data.

In some forms, the Rx data file received in step 52 can be disposed of through permanent deletion, shredding, etc. at step 70, in accord with the applicable policies or legal requirements relating to such data. The operator can also notify the original sender of the Rx data file and/or data custodian that disposal of the Rx data file has occurred, but of course disposal is optional.

Turning now to FIG. 3, one embodiment of the stratification engine is described which is utilized in the stratification engine step 66 discussed above. Once the data has been processed and ingested, each medication listed in a valid Rx claim in the data file is compared against a data listing to determine an associated identifier. The identifier can be determined using a Look Up Table (LUT) at step 72, for example, but other techniques can also be implemented including hosting the identifier in a database and using associated queries to determine the identifier, etc. In one form, the identifier found at step 72 can be related to a National Drug Code (NDC), but in others the identifier can take the form of a propriety identifier other than an identifier associated with an NDC. The identifier determined at step 72 can take a variety of forms including but not limited to alphanumeric form/code. After the identifier has been determined at 72 it can thereafter be added to the data table, database, or other data form which represents the processed data file from step 64. To set forth just one non-limiting example, if the medication found in the Rx claim is clopidogrel, the NDC code of “904629461” can be entered.

After determining the identifier in step 72, a base score (or “composite score”) associated with the individual medication can be determined at step 76 using information already determined from step 72 along with information available after the process at step 74. With particular respect to step 74, a scoring LUT is used to determine the composite scores associated with each medication, where the medication is linked to same identifier used in step 72, where the composite scores are based on information available from a number of different domain sources (including, but not limited to, those describe below). The scoring LUT is therefore populated with composite scores determined from an evaluation of the domains source which are then also associated with each of the identifiers. Data from various domain sources used in the development of the scoring LUT are evaluated to determine a component score for each domain, where the components can then be fused together to form the comprehensive composite score for a particular medication. Fusing/merging the component scores to form the domain score can include any number of different approaches, including but not limited to weighting each component score depending on the importance, credibility, etc of the particular domain prior to summing the weighted components together. In other embodiments, such components may not be weighted. Information can be derived and incorporated into the step at 74 from domain sources such as, but not limited to: (1) population frequency of genetic variation; (2) severity of adverse event; (3) levels of evidence; and (4) actionability (the degree to which genetic characterization correlates to disease risk). Numerical values can be assigned to each of the domain areas, where such values are selected based on perceived, historical, or measured impact in one embodiment, but can be constrained in other embodiments to a value within a range where the range may or may not be dependent upon range(s) assigned with other individual medication(s). In one non-limiting example of the process described immediately above, component scores for each of the domains of the type described above for the drug Clopidogrel can be added to form a composite score of 37.5 out of a maximum of 40.5 based on a particular instance of the weighting algorithms as follows: Evidence: 14.5/14.5—due to warning on FDA label and highest level of evidence in CPIC and PharmGKB guidelines; severity: 7/7—due to severe genetic interactions potentially causing life threatening consequences; actionability: 10/12—due to CPIC guidelines for avoidance and actionable PGx from FDA (not max score as testing not required prior to use); and genetic frequency 6/7—due to high proportion of population with severe and moderate gene-drug interactions for this medication.

The score is open to change over time if input updates to the algorithm are made, such as inputs to any of the associated domain areas. For example, if increasing evidence emerges related to an individual medication then that domain score may change for that particular medication.

Any medication associated with a member within a predetermined time period can have a composite score assigned to that member for that medication. For example, a composite base score can be assigned to the member for a particular medication if that member has had an insurance claim for that medication during the previous 12 months.

At step 78, a member's composite score for any given medication is then time weighted, such as but not limited to weighted based upon length of treatment. Such time weighting can take the form of time-based breakpoints, which decrease in value the longer a member is taking or otherwise has as claim for a particular medication. To set forth an example, a higher weight can be provided the earlier in time a member is taking a medication under the reasoned view that complications arising from a drug are more likely to occur early in treatment. Additionally, the longer a member has been on a medication the more willing healthcare providers, or insurers, may be willing to investigate a different therapy, especially if the current course has proven adequate or the member is generally in a stable state.

Any number of breakpoints can be provided at any number of different time periods. In some forms, the breakpoints can monotonically decrease over time, but other more complex breakpoint shapes can be used. It will be appreciated that the weights and breakpoints can be arbitrarily selected in one embodiment, but can also be constrained in other embodiments to a value within a range where the range may or may not be dependent upon range(s) assigned with other individual medication(s). In one non-limiting example, a member taking clopidogrel for 30 days could have a composite score of 37.5, which then reduces to a composite score of zero for all points in time after 30 days. In another non-limiting example, a member taking clopidogrel for 30 days could have an initial adjusted composite score of 34.9, a time adjusted score composite score of 24.3 for time between 30 days and 180 days, and so on. Any medication that a member has taken in the last 12 months, but the member is not currently taking can be counted as 1/10^{th} of the base composite score, for example. Other values for the breakpoints and weights beyond those described above are contemplated herein.

In another embodiment an exponentially decreasing time-weighting function can be used in lieu of the time-based breakpoints discussed immediately above. The exponential constant can be tailored to provide roughly the same drop off as the breakpoint drops listed immediately above. Such a function provides an analytic determination of the weight and can therefore be implemented to provide a continuous time-weighted function instead of the discrete breakpoint scheme.

At step 80 all of the time-adjusted composite scores for each of the different medications associated with a member are fused together to form a final medication score. Different methods can be used to fuse the data together, including but not limited to summing the various time-adjusted composite scores. Step 82 can also involve adjusting the final medication score based on drug-interactions. An evaluation can be included at step 82 to compare all of the different medications associated with a member, and if adverse interactions are indicated the final medication score can be adjusted (increased, decreased, or remaining the same), either through a scaling factor and/or through a bias. Alternatively and/or additionally, other operations can be used to adjust the final medication score. A random forest model can be used for regression to further adjust the final medication score in the presence of drug-drug interactions. The random forest model can be developed using data collected from patient histories and provided from any variety of sources such as clinical trials, hospital admissions, primary care physician records, etc.

The random forest model is a form of machine learning, or artificial intelligence, that creates multiple decision trees from data samples, the samples often selected randomly, from a larger data or training set, where the final result of the model results from an aggregation or averaging of the individual results of the assembled decisions trees. The random forest model reduces the risk of overfitting the data, provides increased flexibility, and enhances the ability to determine the importance of key features.

Each of the processes at step 82, whether adjusting based on the indication of adverse interactions or the drug-drug interactions derived from the random forest model, can contribute a set amount to the final medication score. In one embodiment each of the separate processes at step 82 can contribute up to 5% of the final medication score, with the other 95% based on the factors described herein, but other values are also contemplated herein.

Step 84 includes normalizing each of the final medication scores for each member to 100 (although other target values could also be used for purposes of normalization). In one form the final medication scores can be normalized using a process that includes, among other things, a log transformation of the final medication score (where a ‘1’ can be used in the log transform for those final medication scores of 0) and in some forms where the base is the maximum final medication score, but other techniques are also contemplated herein. Once normalized and plotted, the final, adjusted medication scores can be clustered into separate risk groups in step 86.

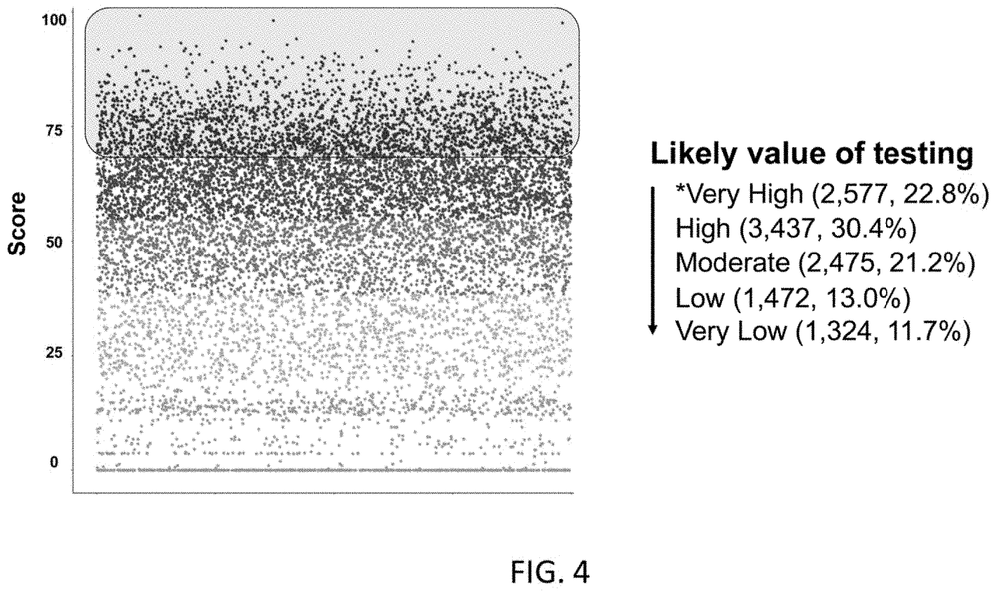

FIG. 4 illustrates one possible stratification clustering for a dataset of time adjusted medication scores. The clustering can be accomplished using a variety of techniques and in one form uses a K-nearest neighbor algorithm in conjunction with an explicit declaration of the n-number of different classes. For example, if five different strata are desired, the K-nearest neighbor algorithm can group data points into the five separate classes by approximately segregating the points into classes that are closest to the mean of each group.

In one embodiment, the strata can be established using the Jenks optimization methods (or Jenks Breaks method). Jenks Breaks is a data clustering methods designed to find the best or most natural breaks, or separation points, in a large data set.

In particular, in FIG. 4 five different strata are indicated by the degree of shading, from the darkest “very high” score group to the lightest “very low” score group. The total number of members in each strata is indicated on the FIG., and the percent of the overall members in the dataset in each group is set forth as well. The “very high” group indicates the members that are most likely to benefit from a PGx test, as they are at the highest risk of being on a non-optimized therapy track. The level of such risk decreases as indicated by the arrow on FIG. 4.

After the different strata have been established, the operator may send a communication to the members in the group(s) identified as the most likely to benefit from PGx testing, or prompt a user to reach out to the members in those groups. Additionally, either the operator or a user may send the members a PGx testing kit to initiate testing.

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected. It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

1. An automated computerized method of data stratification, comprising:
providing a computer processor for carrying out the steps of the method pursuant to executable computer instructions operating on the processor;
inputting a data file for processing by the processor, the data file containing individualized health care data; and
stratifying the data into a plurality of risk groups by determining the likelihood an individual whose data is included in the data file is to benefit from a medical test.

2. The method of claim 1 where the medical test is a pharmacogenomics test.

3. The method of claim 2 further the step of outputting an individualized report or notice identifying the risk level associated therewith.

4. The method of claim 1 further comprising the step of destroying the data file.

5. The method of claim 1 further comprising the step of associating a code with any drug information in the data file.

6. The method of claim 1 further comprising the step of determining one or more composite scores based on information from a variety of sources.

7. The method of claim 6 wherein the variety of sources comprises genetic frequency variation, severity of adverse drug interactions, levels of evidence, and actionability.

8. The method of claim 7 wherein composite scores are determined for each source, and a comprehensive composite score is determined from combining the scores from each source for each medication.

9. The method of claim 6 further comprising the step of adjusting the composite scores by the length of an individual has been taking a particular medication indicated in the data file.

10. The method of claim 6 further comprising the step of adjusting the composite scores based on the length of time an individual has been taking a medication represented in the data file.

11. The method of claim 10 wherein the shorter time on a medication the higher weight given the composite scores.

12. The method of claim 10 where time based break points in the data are determined.

13. The method of claim 10 where a continuous time weighted function is used.

14. The method of claim 6 further comprising the step of combining the composite scores into a single final medication score measuring the cumulative effect of various drugs an individual is using.

15. The method of claim 14 where the combining step uses a random forest model.

16. The method of claim 14 further comprising the step of moralizing the final score.

17. The method of claim 16 where the final scores are the subject of the stratification step.

18. The method of claim 17 where the stratification step uses a nearest neighbor algorithm.

19. The method of claim 17 where the stratification step uses a Jenks Breaks method.

20. The method of claim 17 where 5 different strata are determined.

21. The method of claim 1 further comprising the step of preprocessing the data file to remove errors, redundancies, and correct for formatting.

22. The method of claim 1 where the data file includes individualized demographic data.

23. The method of claim 1 where the source of the data file is a health plan.

24. The method of claim 1 where the source of the data file is a pharmacy.
